(19) United States
(12) Patent Application Publication
(10) Pub. No.: US 2024/0320879 A1
(43) Pub. Date: Sep. 26, 2024

(54) AUTOMATED PARAMETER SELECTION FOR PET SYSTEM

(71) Applicant: GE Precision Healthcare LLC, Wauwatosa, WI (US)

(72) Inventors: Abolfazl Mehranian, Oxford (GB); Scott Wollenweber, Waukesha, WI (US); Kuan-Hao Su, Waukesha, WI (US); Robert John Johnsen, Waukesha, WI (US); Floribertus P Heukensfeldt Jansen, Ballston Lake, NY (US)

(21) Appl. No.: 18/188,377

(22) Filed: Mar. 22, 2023

(51) Int. Cl. G06T 11/00

(52) U.S. Cl. CPC G06T 11/005; G06T 11/008; G06T 2210/41

(57) ABSTRACT

The current disclosure provides systems and methods for increasing a quality of medical images via a recommendation system that recommends appropriate parameter settings to a user of a medical imaging system. In one example, a hybrid recommendation system for recommending a parameter setting for acquiring and/or reconstructing an image via a Positron Emission Tomography (PET) system comprises a first model trained to predict a parameter setting based on a preliminary reconstructed image; and a second model trained to customize the predicted parameter setting based on a preference of a user of the hybrid recommendation system.

### TECHNICAL FIELD

Embodiments of the subject matter disclosed herein relate to positron emission tomography (PET) imaging, and more particularly, to systems and methods for optimizing a quality of PET images.

### BACKGROUND

An image quality (IQ) of medical images may vary based on a set of image acquisition, reconstruction, and processing parameter settings chosen by a user of a medical imaging system. These parameter settings are often selected experimentally based on a clinical task, scanning protocol, scanner specifications, and/or the user's preferences. The settings may vary across patients, where ideal settings or parameters for one patient may not be ideal for a different patient. For example, in Positron Emission Tomography (PET) systems, an acquisition parameter for scan time per bed and a regularization parameter (e.g., a beta value) used for image reconstruction may not be patient specific, as finding appropriate parameter settings for each patient may be time-consuming or clinically impossible. As a result, the parameters used to acquire image data and reconstruct a PET image may be estimated based on trial and error, resulting in images with inconsistent or less than desired quality.

### SUMMARY

The current disclosure at least partially addresses one or more of the above identified issues by a method for a Positron Emission Tomography (PET) system, comprising performing a first reconstruction of a first image volume using raw image data of the PET system without specifying a noise regularization parameter; extracting a plurality of patches of the first image volume; using a first trained model to predict a noise regularization parameter setting, based on the extracted patches; using a second trained model to customize the predicted noise regularization parameter setting for a user of the PET system, based on preference data of the user; performing a second reconstruction of a second image volume using the raw image data of the PET system and the customized, predicted noise regularization parameter setting, the second image volume having a higher image quality than the first image volume; and displaying the second image volume on a display device of the PET system. In various embodiments, the first model is a conventional regression network (CRN) trained on a plurality of images of different noise levels labeled with target parameter settings as ground truth data, and the second model may be a machine learning model, a lookup table, or a different type of model trained on a plurality of images of different noise levels labeled with target parameter settings based on preference data collected from the user. In some embodiments, the first model and the second model may be combined.

In this way, key image quality parameter settings in PET may be identified and parameter recommendation systems may be built that recommend parameter settings tailored to the user's demands and preferences for a desired clinical task (e.g., detectability, quantification, noise reduction, etc.). By using the parameter settings recommended by the parameter recommendation systems, the quality of a resulting image may be higher than the quality of an image reconstructed using parameter settings selected by the user. Additionally, image quality may be more consistent across patients with different demographics.

The above advantages and other advantages, and features of the present description will be readily apparent from the following Detailed Description when taken alone or in connection with the accompanying drawings. It should be understood that the summary above is provided to introduce in simplified form a selection of concepts that are further described in the detailed description. It is not meant to identify key or essential features of the claimed subject matter, the scope of which is defined uniquely by the claims that follow the detailed description. Furthermore, the claimed subject matter is not limited to implementations that solve any disadvantages noted above or in any part of this disclosure.

### BRIEF DESCRIPTION OF THE DRAWINGS

Various aspects of this disclosure may be better understood upon reading the following detailed description and upon reference to the drawings in which:

FIG. 1A is a pictorial view of an exemplary multi-modality imaging system according to one or more embodiments of the disclosure;

FIG. 1B is a block schematic diagram of an exemplary multi-modality imaging system, according to one or more embodiments of the disclosure;

FIG. 2 shows a block diagram of an exemplary embodiment of a parameter recommendation system for a PET imaging system, according to one or more embodiments of the disclosure;

FIG. 3A shows a first block diagram of an exemplary image reconstruction procedure using a recommendation system, according to one or more embodiments of the disclosure;

FIG. 3B shows a second block diagram of an exemplary image reconstruction procedure using a parameter recommendation system, according to one or more embodiments of the disclosure;

FIG. 3C shows a block diagram of an exemplary system for training the parameter recommendation system of FIGS. 3A and 3B, according to one or more embodiments of the disclosure;

FIG. 4 shows example image selections used to train the parameter recommendation system, according to one or more embodiments of the disclosure;

FIG. 5 is a flowchart illustrating an exemplary procedure for using a trained parameter recommendation system to recommend parameter settings for reconstructing a PET image, according to one or more embodiments of the disclosure;

FIG. 6 is a flowchart illustrating an exemplary procedure for training the parameter recommendation system, according to one or more embodiments of the disclosure;

FIG. 7 is a flowchart illustrating an exemplary procedure for training a parameter customization model, according to one or more embodiments of the disclosure;

FIG. 8A shows a first series of exemplary 2D images reconstructed using different beta values, according to one or more embodiments of the disclosure; and

FIG. 8B shows a second series of exemplary 2D images reconstructed using recommended beta values, according to one or more embodiments of the disclosure.

The drawings illustrate specific aspects of the described systems and methods. Together with the following description, the drawings demonstrate and explain the structures, methods, and principles described herein. In the drawings, the size of components may be exaggerated or otherwise modified for clarity. Well-known structures, materials, or operations are not shown or described in detail to avoid obscuring aspects of the described components, systems, and methods.

### DETAILED DESCRIPTION

Systems and methods are disclosed for increasing an image quality of a medical image acquired and reconstructed using a medical imaging system such as Positron Emission Tomography (PET) system. The image quality may depend on a plurality of acquisition and reconstruction parameters that are set or selected by an operator of the PET system prior to or during an exam. Setting the parameters may include performing a first, faster reconstruction method, such as a fast ordered subsets expectation maximization (OSEM) reconstruction, to obtain a preliminary sub-optimal image. For example, the sub-optimal image may include a higher amount of noise than desired. The operator may then adjust one or more acquisition and/or reconstruction parameter settings based on the preliminary image. For example, the operator may specify an acquisition time per bed position for different portions of acquired image data at different positions of a bed of the PET system, based on an amount of noise or a presence of motion in the preliminary noisy image.

As another example, based on the amount of noise in the preliminary noisy image, the operator may specify a noise regularization parameter for reconstructing an image volume from the image data (also referred to herein as a beta value) using a second, slower reconstruction method that may generate a higher quality image volume. The beta value is a de-noising factor of the second reconstruction method, which establishes how close neighboring voxels in the image have to be in terms of absolute value. As the beta value increases, image noise is reduced, but contrast may also be reduced. For longer duration, high dose scans, a level of noise in the image may be reduced and regularization may be less desirable, whereby the beta value may be smaller. For shorter duration, low dose scans, the level of noise in the image may be higher, whereby the beta value may be increased to find a balance between the noise and the signal (contrast). The beta value may also be increased for larger patients, and/or reduced for smaller patients. A visual effect of increasing the beta value may be a smoother image (e.g., less grainy).

However, because the parameter values are selected experimentally, based on the operator's experience in a trial-and-error fashion, a final reconstructed image generated by a second reconstruction method may have a quality (e.g., resolution or contrast-to-noise ratio (CNR)) that is less than desired. The quality of images acquired and reconstructed by different operators may also vary. To increase the quality and consistency of the images, the inventors herein propose a parameter recommendation system for the PET system, where the parameter recommendation system takes as input the preliminary noisy image and the initial parameters, and recommends one or more parameter settings to achieve a desired image quality. The parameter recommendation system is described herein with respect to recommending an appropriate beta value for performing a scan. However, it should be appreciated that in other embodiments, the parameter recommendation system described herein could be used to recommend other parameter settings.

Additionally, the proposed recommendation system may be a hybrid recommendation system, including a first model trained to output a recommended parameter based on objective criteria, such as a scanning protocol, scanner specifications, a desired clinical task, a size of a scanned subject, and the like; and a second model trained to customize or fine tune the recommended parameter outputted by the first model based on subjective preferences of the operator, such as a degree of comfort with respect to a level of noise or desired smoothness of the reconstructed image. The hybrid RS may comprise multiple recommender models, including collaborative systems, content-based filtering techniques, and/or machine learning models, which may learn the IQ parameter settings based on users' inputs and customize the parameter settings for an individual patient or group of patients. The inputs may include, for example, ratings of various images, available settings, and settings preferred by a specific user or various users. The recommendation system may further take into account objective criteria for achieving certain objectives, for example, “detecting lesions of a certain size above the noise”, and optimizing acquisition and reconstruction parameters in order to maximize detectability or other measure that scales with the success of the task in hand.

In some cases, the recommender system may trigger data-driven motion tracking for a given bed position, based on an anatomy of a subject and a presence of lesions. The recommender system may leverage prior exams and reports, or rely on CT scans acquired during a same scanning session. The recommender system may use natural language processing (NLP) as well as convolutional neural networks for CT image segmentation. Further, when motion correction is enabled for a bed during acquisition, a gated motion compensation technique may be used to reduce the total counts of the bed. The reduction of counts can be compensated by extending the scanning duration. In this situation, the scanning duration can be automatically extended for a certain amount of time by the recommender system based on an image quality preference of a user.

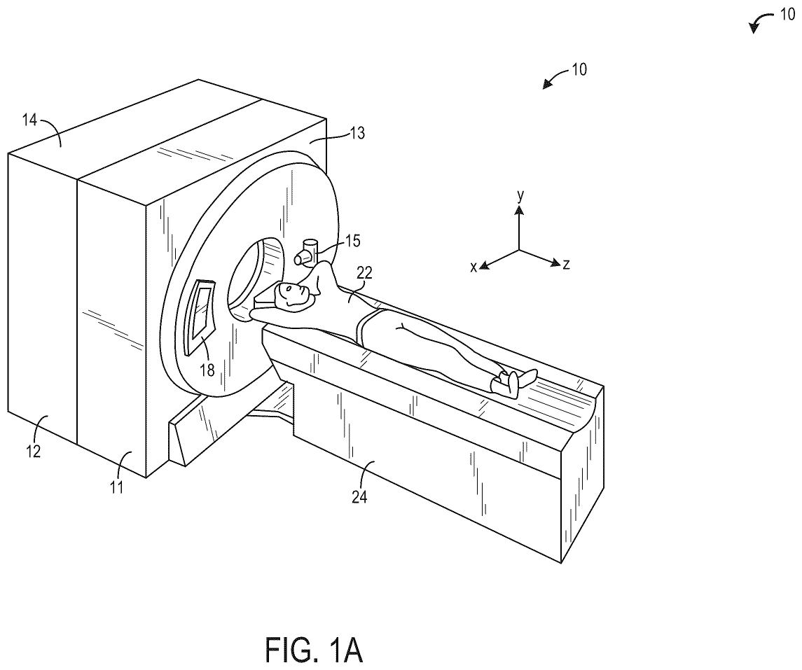

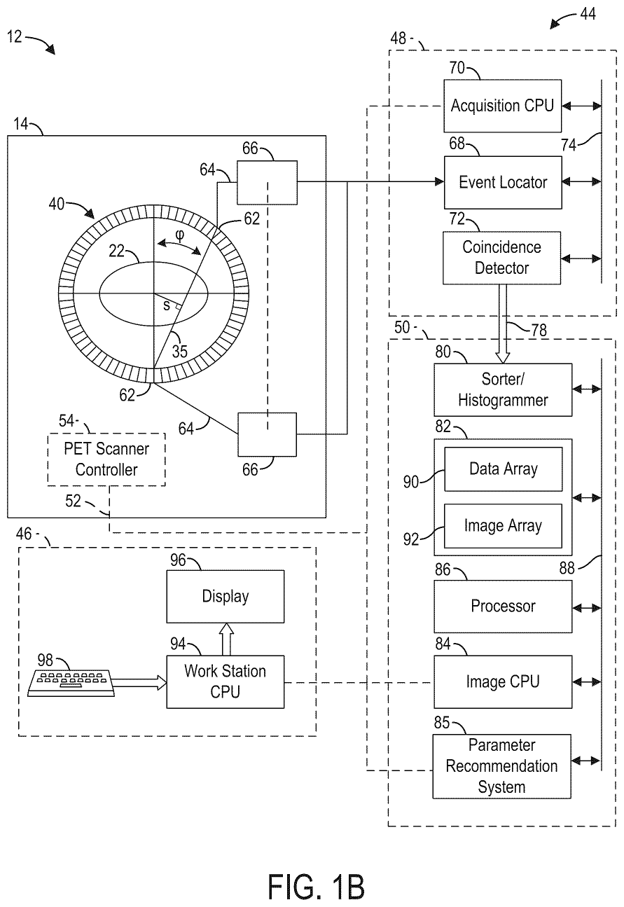

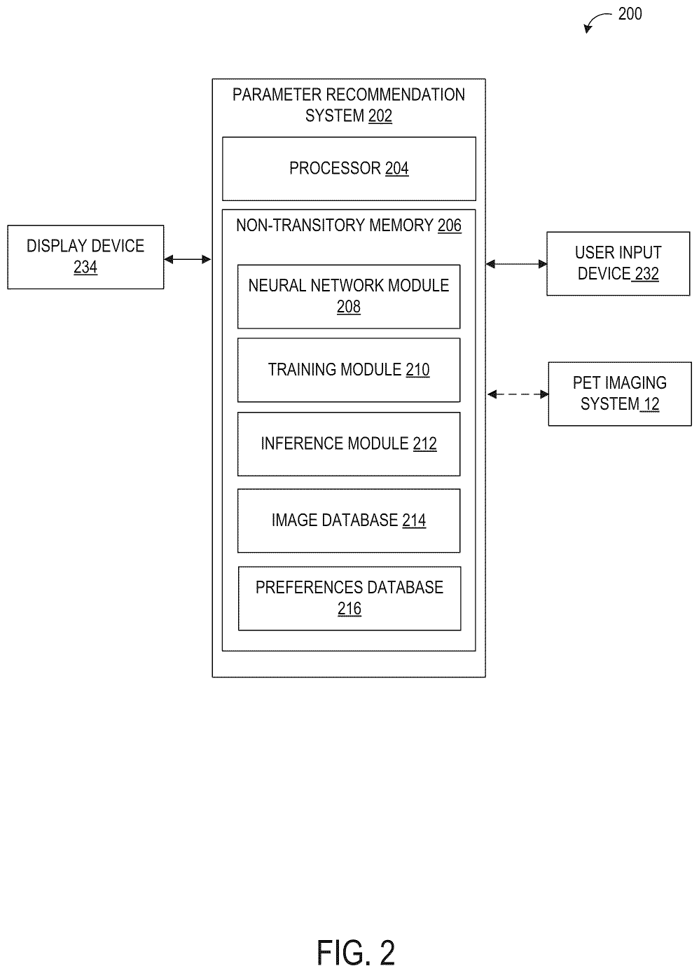

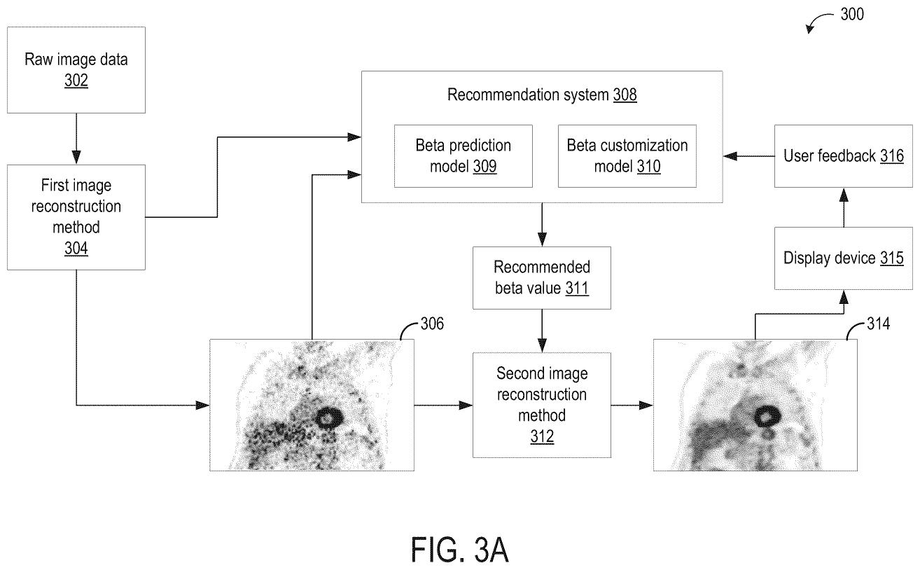

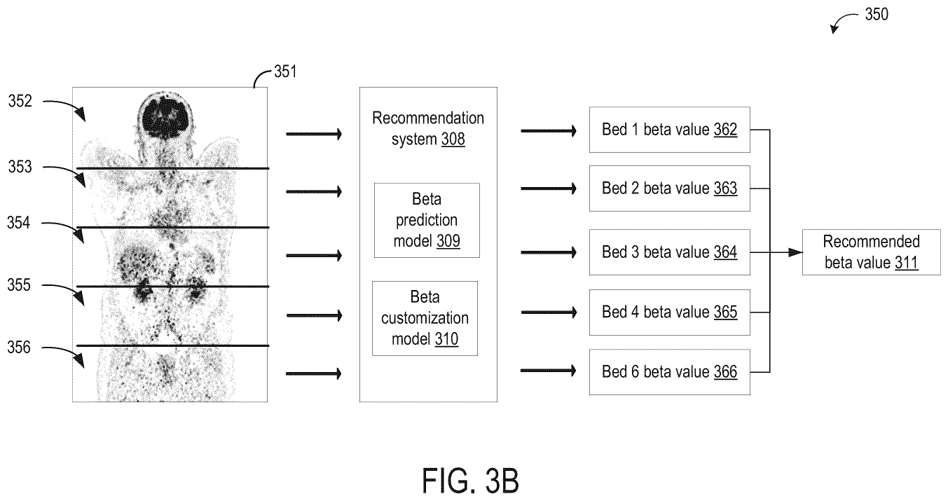

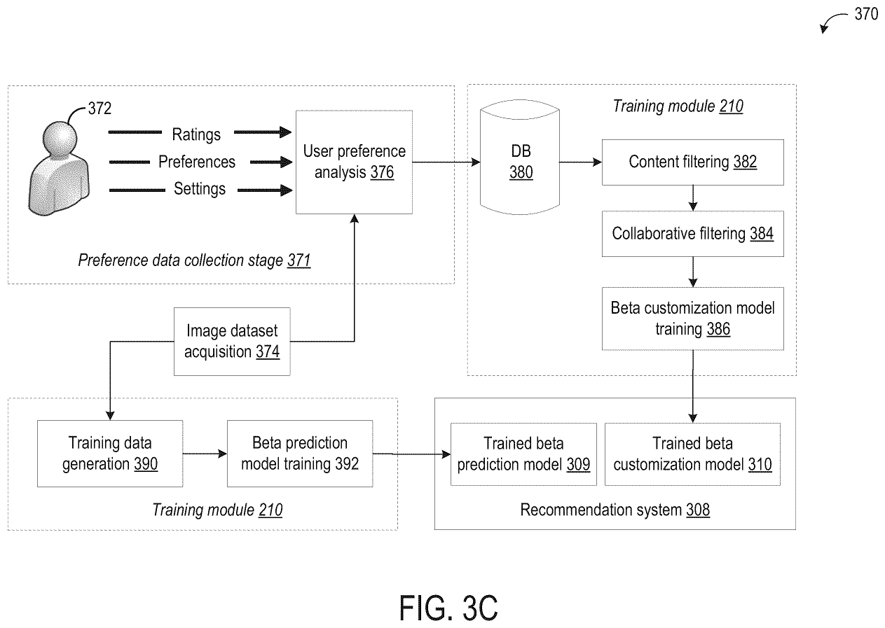

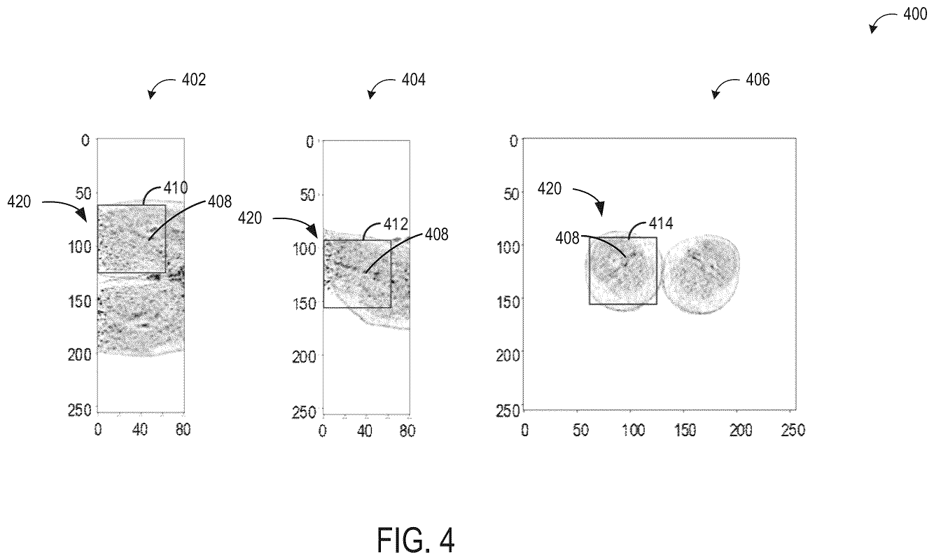

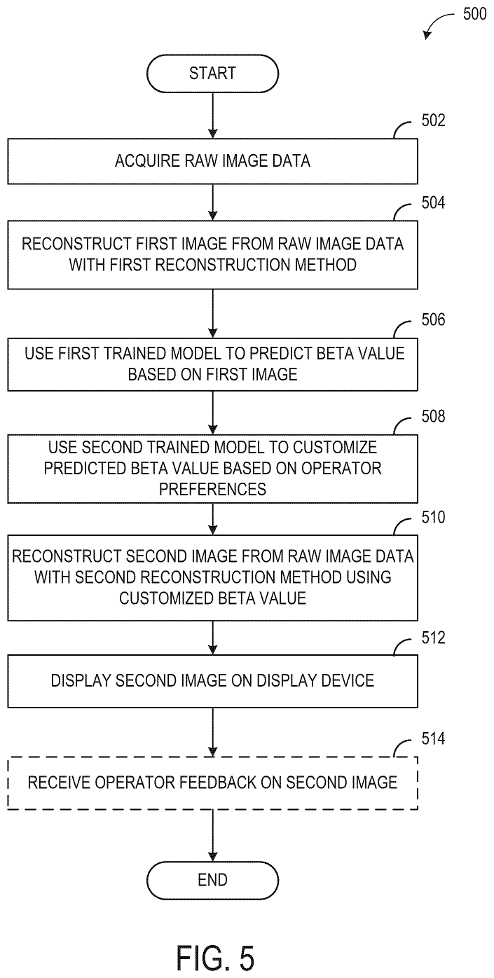

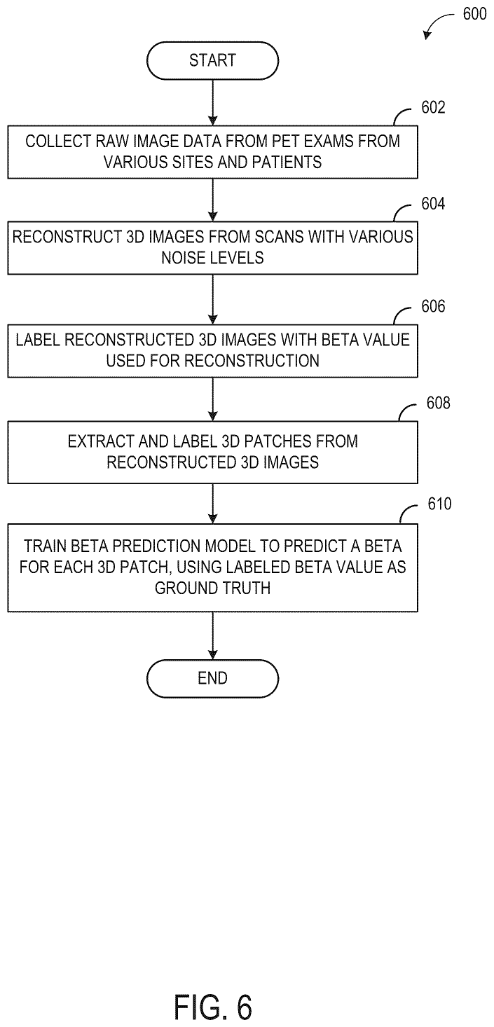

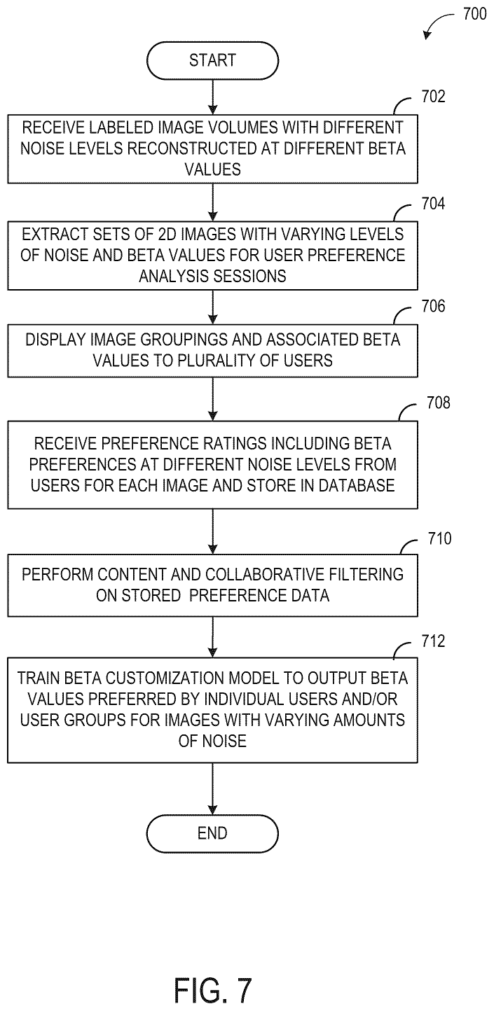

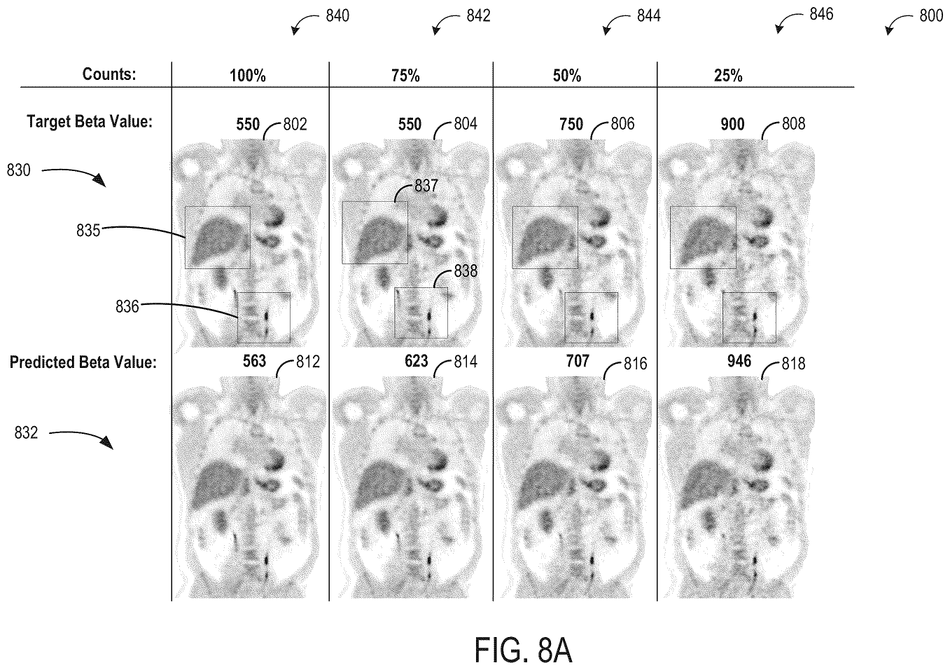

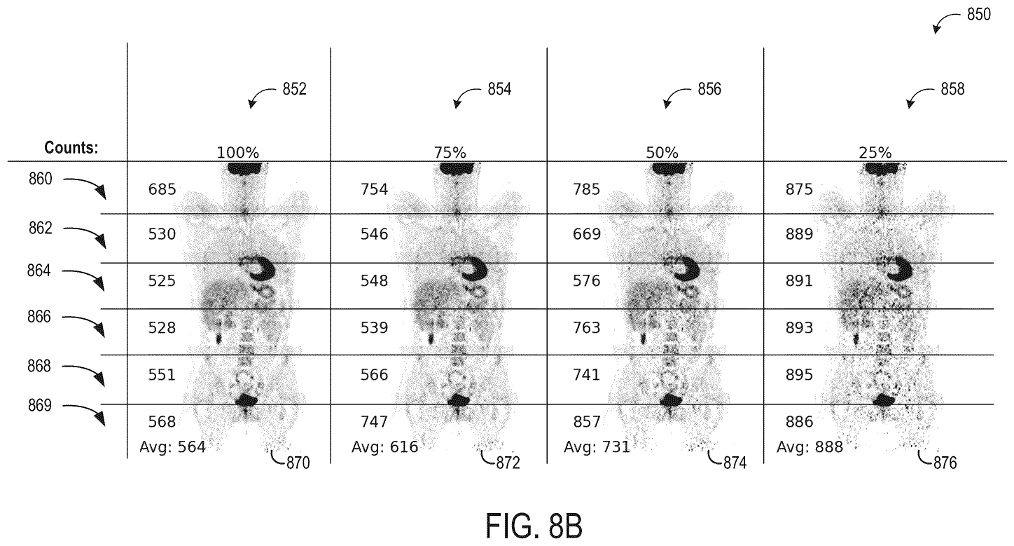

The medical imaging system may be a multi-modality imaging system including PET imaging capabilities, such as the multi-modality imaging system shown in FIGS. 1A and 1B. The multi-modality imaging system may include a parameter recommendation system, such as the parameter recommendation system shown in FIG. 2. In one embodiment, the parameter recommendation system may be used to recommend a beta value for image reconstruction, as shown in FIG. 3A. The parameter recommendation system may be used to recommend different beta values for different bed positions of the multi-modality imaging system, as shown in FIG. 3B. The parameter recommendation system may include a beta prediction model and a beta customization model, which may be trained as shown in FIG. 3C and as described in reference to the method of FIG. 6. The beta prediction model may be trained on image patches selected from one or more reconstructed images, as shown in FIG. 4. In some embodiments, the beta prediction model may be a convolutional neural network (CNN). The beta customization model may be trained on images rated by one or more operators, by following one or more steps of the method shown in FIG. 7. Once the beta prediction model and beta customization model are trained, the parameter recommendation system may be used to recommend a beta value for image reconstruction during operation of the multi-modality imaging system by following one or more steps of the method shown in FIG. 5. The parameter recommendation system may recommend parameter settings that are similar to settings preferred by operators, as shown in FIG. 8A, and may further recommend appropriate parameter settings for different bed positions, as shown in FIG. 8B.

Various embodiments of the disclosure provide a multi-modality imaging system 10 as shown in FIGS. 1A and 1B. Multi-modality imaging system 10 may be any type of imaging system, for example, different types of medical imaging systems, such as a Positron Emission Tomography (PET), a Single Photon Emission Computed Tomography (SPECT), a Computed Tomography (CT, an ultrasound system, Magnetic Resonance Imaging (MRI), or any other system capable of generating tomographic images. The various embodiments are not limited to multi-modality medical imaging systems, but may be used on a single modality medical imaging system such as a stand-alone PET imaging system or a stand-alone SPECT imaging system, for example. Moreover, the various embodiments are not limited to medical imaging systems for imaging human subjects, but may include veterinary or non-medical systems for imaging non-human objects. In general, while the disclosed materials are described herein in reference to a PET imaging system, it should be appreciated that other types of imaging systems may be used without departing from the scope of this disclosure.

Referring to FIG. 1A, the multi-modality imaging system 10 includes a first modality unit 11 and a second modality unit 12. The two modality units enable the multi-modality imaging system 10 to scan an object or patient in a second modality using the second modality unit 12. The multi-modality imaging system 10 allows for multiple scans in different modalities to facilitate an increased diagnostic capability over single modality systems. In one embodiment, multi-modality imaging system 10 is a Computed Tomography/Positron Emission Tomography (CT/PET) imaging system 10, e.g., the first modality 11 is a CT imaging system 11 and the second modality 12 is a PET imaging system 12. The CT/PET system 10 is shown as including a gantry 13 representative of a CT imaging system and a gantry 14 that is associated with a PET imaging system. As discussed above, modalities other than CT and PET may be employed with the multi-modality imaging system 10.

The gantry 13 includes an x-ray source 15 that projects a beam of x-rays toward a detector array 18 on the opposite side of the gantry 13. Detector array 18 is formed by a plurality of detector rows (not shown) including a plurality of detector elements which together sense the projected x-rays that pass through a patient 22. Each detector element produces an electrical signal that represents the intensity of an impinging x-ray beam and hence allows estimation of the attenuation of the beam as it passes through the patient 22. During a scan to acquire x-ray projection data, gantry 13 and the components mounted thereon rotate about a center of rotation.

FIG. 1B is a block schematic diagram of the PET imaging system 12 illustrated in FIG. 1A in accordance with an embodiment of the present disclosure. The PET imaging system 12 includes a detector ring assembly 40 including a plurality of detector crystals. The PET imaging system 12 also includes a controller or processor 44, to control normalization, image reconstruction processes and perform calibration. Controller 44 is coupled to an operator workstation 46. Controller 44 includes a data acquisition processor 48 and an image reconstruction processor 50, which are interconnected via a communication link 52. PET imaging system 12 acquires scan data and transmits the data to data acquisition processor 48. The scanning operation is controlled from the operator workstation 46. The data acquired by the data acquisition processor 48 is reconstructed using the image reconstruction processor 50.

When performing a scan, an operator using operator workstation 46 may set various acquisition parameters used by data acquisition processor 48 for acquiring the data, and/or various reconstruction parameters used by image reconstruction processor 50 for reconstructing an image based on the acquired data. The acquisition and parameter settings may differ depending on a clinical task, a selected scanning protocol, specifications of PET imaging system 12, and/or other factors. The acquisition and parameter settings may also vary across patients, depending on a size, age, or other characteristics of a patient. For example, a first regularization parameter used for image reconstruction (e.g., beta value) may be used for a first, smaller patient, and a second regularization parameter may be used for a second, larger patient. The acquisition and parameter settings may be selected by the operator based on operator experience and/or trial and error. For example, one or more preliminary (fast) reconstructions may be performed to test appropriate beta values to use, and an appropriate beta value may be selected for a second, higher quality image reconstruction. As another example, an acquisition parameter for a scan time per bed during data acquisition may be determined by a trial-and-error process, where various data acquisitions are performed to test different settings for the acquisition parameter prior to acquiring data for reconstruction.

The detector ring assembly 40 includes a central opening, in which an object or patient, such as patient 22 may be positioned using, for example, a motorized table or bed 24 (shown in FIG. 1A). The motorized table 24 is aligned with the central axis of detector ring assembly 40. This motorized table 24 moves the patient 22 into the central opening of detector ring assembly 40 in response to one or more commands received from the operator workstation 46. A PET scanner controller 54, also referred to as the PET gantry controller, is provided (e.g., mounted) within PET system 12. The PET scanner controller 54 responds to the commands received from the operator workstation 46 through the communication link 52. Therefore, the scanning operation is controlled from the operator workstation 46 through PET scanner controller 54.

During operation, when a photon collides with a crystal 62 on a detector ring 40, it produces a scintillation event on the crystal. One or more photosensors are coupled to the scintillation crystals, and produce a signal in response to the scintillation that may be transmitted on communication line 64. A set of acquisition circuits 66 is provided to receive these signals. Acquisition circuits 66 convert these signals to indicate the three-dimensional (3D) location, timing, and total energy of the event. These digital signals are transmitted through a communication link, for example, a cable, to an event locator circuit 68 in the data acquisition processor 48.

The data acquisition processor 48 includes the event locator circuit 68, an acquisition CPU 70, and a coincidence detector 72. The data acquisition processor 48 periodically samples the signals produced by the acquisition circuits 66. The acquisition CPU 70 controls communications on a back-plane bus 74 and on the communication link 52. The event locator circuit 68 processes the information regarding each valid event and provides a set of digital numbers or values indicative of the detected event. For example, this information indicates when the event took place and the position of the scintillation crystal 62 that detected the event. An event data packet is communicated to the coincidence detector 72 through the back-plane bus 74. The coincidence detector 72 receives the event data packets from the event locator circuit 68 and determines if any two of the detected events are in coincidence. Coincidence is determined by a number of factors. First, the time markers in each event data packet must be within a predetermined time period, for example, 5.3 nanoseconds, of each other. Second, the line-of-response (LOR) formed by a straight line joining the two detectors that detect the coincidence event should pass through the field of view in the PET imaging system 12. Events that cannot be paired are discarded. Coincident event pairs are located and recorded as a coincidence data packet that is communicated through a physical communication link 78 to a sorter/histogrammer 80 in the image reconstruction processor 50.

The image reconstruction processor 50 includes the sorter/histogrammer 80. During operation, sorter/histogrammer 80 generates a data structure known as a histogram. A histogram includes a large number of cells, where each cell corresponds to a pair of detector crystals in the PET scanner. Because a PET scanner typically includes thousands of detector crystals, the histogram typically includes millions of cells. Each cell of the histogram also stores a count value representing the number of coincidence events detected by the pair of detector crystals for that cell during the scan. At the end of the scan, the data in the histogram is used to reconstruct an image of the patient. The completed histogram containing all the data from the scan is commonly referred to as a “sinogram.” The term “histogrammer” generally refers to the components of the scanner, e.g., processor and memory, which carry out the function of creating the histogram.

The image reconstruction processor 50 also includes a memory module 82, an image CPU 84, an array processor 86, and a communication bus 88. During operation, the sorter/histogrammer 80 counts all events occurring along each projection ray and organizes the events into 3D data. This 3D data, or sinogram, is organized in one exemplary embodiment as a data array 90. Data array 90 is stored in the memory module 82. The communication bus 88 is linked to the communication link 52 through the image CPU 84. The image CPU 84 controls communication through communication bus 88. The array processor 86 is also connected to the communication bus 88. The array processor 86 receives data array 90 as an input and reconstructs images in the form of image array 92. Resulting image arrays 92 are then stored in memory module 82.

The images stored in the image array 92 are communicated by the image CPU 84 to the operator workstation 46. The operator workstation 46 includes a CPU 94, a display 96, and an input device 98. The CPU 94 connects to communication link 52 and receives inputs, e.g., user commands, from the input device 98. The input device 98 may be, for example, a keyboard, mouse, a touch-screen panel, and/or a voice recognition system, and so on. Through input device 98 and associated control panel switches, the operator can control the operation of the PET imaging system 12 and the positioning of the patient 22 for a scan. Similarly, the operator can control the display of the resulting image on the display 96 and can perform image-enhancement functions using programs executed by the workstation CPU 94.

Additionally, as described in greater detail herein, PET imaging system 12 may include a parameter recommendation system 85 that may suggest appropriate acquisition and/or reconstruction parameter settings to the operator via workstation CPU 94 and/or display 96. For example, in one embodiment, a first, fast reconstruction may be performed without specifying a beta value, and a resulting image may be inputted into parameter recommendation system 85. The fast reconstruction may provide information about noise without regularization which may help determine a desired amount of regularization (e.g., an appropriate beta value) to be applied to reconstruct a final image. Parameter recommendation system 85 may suggest a beta value to use for the final reconstruction, based on the resulting image from the fast reconstruction. The suggested beta value may be recommended based on an output of one or more models (e.g., statistical models, AI models, neural network models, machine learning models, etc.) included in parameter recommendation system 85. The one or more models may be trained to predict a most appropriate beta value based on preferences of the operator, as described in greater detail below.

The detector ring assembly 40 includes a plurality of detector units. The detector unit may include a plurality of detectors, light guides, scintillation crystals and application specific integrated chips (ASICs), which may be analog, digital, or hybrid. For example, the detector unit may include twelve SiPM devices, four light guides, 144 scintillation crystals, and two analog ASICs.

Referring to FIG. 2, a block diagram 200 shows an example of a parameter recommendation system 202, in accordance with an embodiment, where parameter recommendation system 202 may recommend settings for one or more parameters used during an exam performed using a PET imaging system such as PET imaging system 12 described above in reference to FIGS. 1A and 1B. As such, parameter recommendation system 202 may be a non-limiting example of parameter recommendation system 85 of FIG. 1B. In some embodiments, parameter recommendation system 202 is incorporated into the PET imaging system, as described above. For example, parameter recommendation system 202 may rely on processor 116 and memory 120. In some embodiments, at least a portion of parameter recommendation system 202 is disposed at a device (e.g., workstation, edge device, server, etc.) communicably coupled to the PET imaging system via wired and/or wireless connections, which can receive images from the PET imaging system or from a storage device which stores the images/data generated by the PET imaging system. Parameter recommendation system 202 may be operably/communicatively coupled to a user input device 232 and a display device 234. User input device 232 may comprise the user interface 115 of the PET imaging system 12, while display device 234 may comprise the display device 118 of the PET imaging system 12, at least in some examples.

Parameter recommendation system 202 includes a processor 204 configured to execute machine readable instructions stored in non-transitory memory 206. Processor 204 may be single core or multi-core, and the programs executed thereon may be configured for parallel or distributed processing. In some embodiments, processor 204 may optionally include individual components that are distributed throughout two or more devices, which may be remotely located and/or configured for coordinated processing. In some embodiments, one or more aspects of processor 204 may be virtualized and executed by remotely-accessible networked computing devices configured in a cloud computing configuration.

Non-transitory memory 206 may store a neural network module 208, a training module 210, an inference module 212, an image database 214, and a preferences database 216. Neural network module 208 may include at least a neural network model, such as a convolutional neural network (CNN), and instructions for implementing the neural network model to predict one or more parameter settings for the PET imaging system, as described in greater detail below. Neural network module 208 may include trained and/or untrained neural networks and may further include various data, or metadata pertaining to the one or more neural networks stored therein.

Training module 210 may comprise instructions for training one or more of the neural networks stored in neural network module 208 and/or other types of artificial intelligence (AI), machine learning, statistical, or other models. Training module 210 may include instructions that, when executed by processor 204, cause parameter recommendation system 202 to conduct one or more of the steps of method 600 for training a neural network model, discussed in more detail below in reference to FIG. 6. In some embodiments, training module 210 may include instructions for implementing one or more gradient descent algorithms, applying one or more loss functions, and/or training routines, for use in adjusting parameters of one or more neural networks of neural network module 208. Training module 210 may include training datasets for the one or more neural networks of neural network module 208.

Inference module 212 may include instructions for deploying a trained model, for example, to predict PET system acquisition and/or reconstruction settings as described below with respect to FIG. 5. In particular, inference module 212 may include instructions that, when executed by processor 204, cause parameter recommendation system 202 to conduct one or more of the steps of method 500, as described in further detail below.

Image database 214 may include, for example, PET images acquired via the PET imaging system. Image database 214 may include two dimensional (2D) or three dimensional (3D) PET images used in one or more training sets for training the one or more neural networks of neural network module 208.

Preferences database 216 may include data regarding preferences of various users of parameter recommendation system 202 with respect to one or more parameter settings used when performing a scan with the PET imaging system. For example, a quality of an image reconstructed based on the scan may depend on a duration of the scan, or an injected dose of a tracer. A longer duration scan may generate a higher quality image than a shorter duration scan, and a higher injected dose may generate a higher quality image than a smaller injected dose. However, the higher injected dose may expose a subject of the scan to an increased amount of radiation with respect to the lower injected dose, and a longer duration scan may be less comfortable for the patient than a shorter duration scan. Therefore, a trade-off may exist between an amount of radiation to which the subject is exposed, a duration of a scan, and an acceptable amount of noise in the reconstructed image. A first physician desiring a higher-quality image may choose to perform the longer duration scan, thereby reducing a comfort level of the patient, or choose to increase the injected dose, thereby exposing the subject to the increased amount of radiation. A second physician may be more comfortable viewing noisy images, and may choose to perform the shorter duration scan to increase the comfort level of the patient, and/or use a smaller injected dose, to decrease the amount of radiation to which the subject is exposed. Thus, the first physician may have a preference for higher-count exams with lower beta values, and the second physician may have a preference for lower-count exams with higher beta values. The preferences of different users of parameter recommendation system 202 may be collected manually, and stored in preferences database 216. The preferences may be accessed from preferences database 216 by a beta customization model of parameter recommendation system 202, as described in greater detail below in reference to FIG. 3C and FIG. 7.

User input device 232 may comprise one or more of a touchscreen, a keyboard, a mouse, a trackpad, a microphone, a motion sensing camera, or other device configured to enable a user to interact with and manipulate data within parameter recommendation system 202. In one example, user input device 232 may enable a user to make a selection of an image to use in training a model, or for further processing using a trained model.

Display device 234 may include one or more display devices utilizing virtually any type of technology. In some embodiments, display device 234 may comprise a computer monitor, and may display 2D PET images. Display device 234 may be combined with processor 204, non-transitory memory 206, and/or user input device 232 in a shared enclosure, or may be peripheral display devices and may comprise a monitor, touchscreen, projector, or other display device known in the art, which may enable a user to view images produced by an PET imaging system, and/or interact with various data stored in non-transitory memory 206.

It should be understood that parameter recommendation system 202 shown in FIG. 2 is for illustration, not for limitation. Another appropriate parameter recommendation system may include more, fewer, or different components.

FIG. 3A shows a block diagram 300 of an exemplary image reconstruction process using a recommendation system 308, which may be a non-limiting example of parameter recommendation system 202 of FIG. 2. The image reconstruction may begin with raw image data 302, which may be acquired by a PET imaging system such as PET imaging system 12 of FIG. 1B. A first image reconstruction method 304 may be used to reconstruct a preliminary image 306, without specifying initial reconstruction parameters such as a beta value (e.g., noise regularization parameter). First image reconstruction method 304 may be a fast reconstruction method with a first, shorter duration, such as a fast OSEM reconstruction.

Preliminary image 306 may be inputted into recommendation system 308. Recommendation system 308 may include a plurality of models, which may generate a recommended beta value 311. Recommended beta value 311 may then be used by a second image reconstruction method 312 to reconstruct a final reconstructed image 314, which may be displayed on a display device 315 (e.g., display device 234 of FIG. 2). As a result of using recommended beta value 311, final reconstructed image 314 may have a higher image quality (e.g., less noise, a higher resolution, and/or a higher contrast to noise ratio (CNR)) than preliminary image 306. Additionally, a duration of second image reconstruction method 312 may be longer than a duration of first image reconstruction method 304, thereby increasing the quality of final reconstructed image 314 with respect to preliminary image 306.

Recommendation system 308 may be a hybrid recommendation system including a plurality of different types of models, depending on the parameter and/or embodiment. In the depicted embodiment, recommendation system 308 includes a beta prediction model 309 and a beta customization model 310. In the depicted embodiment, preliminary image 306 is inputted into beta prediction model 309. Beta prediction model 309 may be trained to suggest an appropriate beta value based on preliminary image 306, as described in greater detail below in reference to FIG. 6. The beta value suggested by beta prediction model 309 may then be inputted into beta customization model 310. Beta customization model 310 may be trained to customize the suggested beta value based on operator preferences, as described in greater detail below in reference to FIG. 7. Recommended beta value 311 may be the customized beta value outputted by beta customization model 310.

When final reconstructed image 314 is displayed on display device 315, in some embodiments, the operator may provide user feedback 316 with respect to the image quality of final reconstructed image 314. For example, the operator may rate the image quality of final reconstructed image 314, or specify that recommended beta value 311 be adjusted to increase a quality of final reconstructed image 314. The user feedback 316 may be used to further train beta customization model 310 and/or beta prediction model 309.

FIG. 3B shows an embodiment 350 of recommendation system 308 configured to recommend different beta values for different portions of raw image data acquired at a different positions of a bed of the PET imaging system (e.g., table 24 of FIG. 1A). For example, the acquisition may be divided into five different bed positions. Thus, an image 351 reconstructed by the PET imaging system may include a first portion 352 from a first bed position, a second portion 353 from a second bed position, a third portion 354 from a third bed position, a fourth portion 355 from a fourth bed position, and a fifth portion 356 from a fifth bed position. When recommendation system 308 receives image 351, recommendation system 308 may partition image 351 in accordance with the five bed positions, and input image data from each of the portions into beta prediction model 309 and/or beta customization model 310 separately. Beta prediction model 309 and/or beta customization model 310 may predict an appropriate beta value for each portion separately. The input image data from the different portions may comprise patches extracted from image (volume) 351.

For example, a first patch may be extracted from first portion 352 of image 351 and inputted into beta prediction model 309, which may output a first recommended beta value for first portion 352. The recommended beta value for first portion 352 may be inputted into beta customization model 310, which may output a recommended bed 1 beta value 362 for the operator for first portion 352. A second patch may be extracted from second portion 353 and inputted into beta prediction model 309, which may output a second recommended beta value for second portion 353. The recommended beta value for second portion 353 may be inputted into beta customization model 310, which may output a recommended bed 2 beta value 363 for the operator for second portion 353. A third patch may be extracted from third portion 354 and inputted into beta prediction model 309, which may output a third recommended beta value for third portion 354. The recommended beta value for third portion 354 may be inputted into beta customization model 310, which may output a recommended bed 3 beta value 364 for the operator for third portion 354, and so on, to generate a recommended bed 4 beta value 365 for fourth portion 355 and a recommended bed 5 beta value 366 for fifth portion 356. The recommended beta values 362-366 may each be different, or some or all of the recommended beta values may be the same.

In some embodiments, the recommended beta values 362-366 may be combined to generate recommended beta value 311, and recommended beta value 311 may be used to reconstruct final reconstructed image 314 of FIG. 3A. For example, recommended beta value 311 may be a weighted average of the recommended beta values 362-366, where weights for each of recommended beta values 362-366 may be assigned or selected by the operator, or in a different way. In other embodiments, different portions of final reconstructed image 314 (corresponding to the five bed positions) may be reconstructed using the different recommended beta values 362-366. For example, the operator may have a first beta value preference for first portion 352; a second beta value preference for second portion 353; a third beta value preference for third portion 354; and so on. As a result of reconstructing the different portions of final reconstructed image 314 with the different recommended beta values 362-366, the operator may more clearly view features or structures of selected regions of interest of a scanned subject, which may lead to better patient outcomes.

FIG. 3C shows a block diagram of an exemplary training system 370 for training beta prediction model 309 and beta customization model 310 of parameter recommendation system 308 of FIGS. 3A and 3B. Training of beta prediction model 309 and beta customization model 310 may be performed within a training module of parameter recommendation system 308, such as training module 210 of FIG. 2. Training of beta prediction model 309 and beta customization model 310 may be performed separately.

Training of beta prediction model 309 and beta customization model 310 may begin by acquiring a plurality of image datasets via an image dataset acquisition block 374. For example, the image datasets may be generated from a plurality of imaging exams. In some examples, the image datasets may be stored in or drawn from a database of stored PET images (e.g., image database 214 of FIG. 2). From the image datasets, training data for beta prediction model 309 may be generated at a training data generation block 390. The training data may be used to train beta prediction model 309 at a beta prediction model training block 392. Generation of the training data and the training of beta prediction model 309 is described in greater detail below in reference to FIG. 6.

Images from the image datasets acquired at image dataset acquisition block 374 may also be used to perform a user preference analysis 376, during a preference data collection stage 371. During the user preference analysis 376, images may be displayed to one or more users 372, where the one or more users 372 are operators of the PET imaging system and/or recommendation system 308. For example, the one or more users 372 may be radiologists or physicians skilled in reading PET images. The one or more users 372 may rate the images based on personal preference for noise characteristics of the images. For example, some users may prefer images generated from longer scans with less noise and a higher CNR, while other users may prefer shorter scans, and may be comfortable viewing images with greater amounts of noise and/or lower CNRs. In addition to rating the images, the users may also provide preferential settings of various acquisition and/or reconstruction parameters (such as the beta value).

In one example, during training, the users are shown pairs of images in multiple forced-choice pairs and asked to express a preference. From the multiple forced-choice pairs, a ranking may be generated and an optimum may be found. These preferences could further be learned by a retrospective survey following an exam, with the user rating the acquired exam, with the results being used to inform futher analysis. The collection of user preference data from the users is described in greater detail below in reference to FIG. 7.

The user preference data collected from the user preference analysis 376 may be stored in a user preference database 380, where it may be accessed for training beta customization model 310. The user preference data may be processed, for example, in one or both of a content filtering stage 382 and a collaborative filtering stage 384. Content filtering may be based on the preferences of a single user, while collaborative filtering may be based on the preferences of a plurality of users. During content filtering stage 382 and/or collaborative filtering stage 384, the user preference data may be grouped into various categories or classifications by a clustering algorithm or similar statistical methods. For example, users with similar preferences may be classified into a same group.

Once the user preference data has been processed, beta customization model 310 may be trained at a beta customization training block 386. Training of beta customization model 310 on the processed user preference data is described in greater detail below in reference to FIG. 7.

Referring now to FIG. 5, an exemplary method 500 is shown for using a parameter recommendation system (e.g., parameter recommendation system 202 of FIG. 2 and/or parameter recommendation system 308 of FIG. 3A) to recommend one or more parameter settings to an operator of a multi-modality imaging system, such as PET imaging system 12 of FIG. 1B. Method 500 may be carried out by an inference module of the parameter recommendation system, such as inference module 212 of parameter recommendation system 202. One or more instructions of method 500 may be executed by a processor of the parameter recommendation system (e.g., processor 204) based on instructions stored in the inference module. One or more steps of method 500 may be performed manually by the operator.

Method 500 begins at 502, where method 500 includes acquiring raw image data for image reconstruction. The raw image data may be acquired via the PET imaging system. For example, an operator of the PET imaging system (e.g., a physician or technician) may acquire the raw image data via the PET imaging system using a workstation of the PET imaging system (e.g., operator workstation 46 of FIG. 1B).

At 504, method 500 includes reconstructing a first image volume from the raw image data received at 502, using a first reconstruction method without specifying a beta value. The first reconstruction method may be a fast reconstruction method, such as fast OSEM reconstruction. The first reconstruction method may be performed with the aim of rapidly generating a preliminary, sub-optimal image volume. The preliminary, sub-optimal image volume may be used as an aid in determining a suitable beta value for performing a subsequent reconstruction. The first reconstruction may be performed using standard or default parameter settings for scanner type, protocol, tracer type, clinical task, etc.

In some embodiments, the reconstruction of the first image volume may be performed by the operator via the workstation prior to launching or opening the parameter recommendation system on the workstation. The first image volume may be displayed on a display of the workstation (e.g., display 96). The operator may then open the parameter recommendation system and submit the first image volume to the parameter recommendation system manually. In various embodiments, the parameter recommendation system may be automatically launched or opened, and the first image volume may be submitted to the parameter recommendation system without operator input. In some cases, the first image volume may not be displayed on the display of the workstation.

At 506, method 500 includes using a first trained model to recommend a second beta value, based on the first, preliminary noisy image volume. The first trained model may be a beta prediction model, such as beta prediction model 309 of FIG. 3A. In various embodiments, the first trained model may be a neural network model such as a CNN. In various embodiments, one or more patches may be extracted from the first, preliminary noisy image volume. For example, the patches may be 2D patches, or 3D patches. The patches may be inputted into an input layer of the neural network model. For example, image data of a first voxel of a first patch may be inputted into a first input node of the neural network model; image data of a second voxel of the first patch may be inputted into a second input node of the neural network model; image data of a third voxel of the first patch may be inputted into a third input node of the neural network model; and so on. Other characteristics and/or settings of the PET imaging system may also be inputs into the neural network model, such as the selected scanning protocol, type of scanner, anatomical region of interest, clinical task, etc.

The input data for each patch may be propagated through the neural network model, and the neural network model may output predicted beta values corresponding to the patch. The parameter recommendation system may then combine the predicted beta values for each patch to generate a predicted beta value for the first image volume. In one embodiment, the predicted beta value for the first image volume is a median predicted beta value of the plurality of patches. In other embodiments, the predicted beta value for the first image volume may be an average predicted beta value of the plurality of patches, or a weighted average (for example, based on a prioritization of a specific region), or the predicted beta value for the first image volume may be calculated in a different way. The predicted beta value for the first image volume may be different from the first beta value used in the first reconstruction. The predicted beta value may be a recommended setting for reconstructing a second image volume of higher quality than the first image volume.

Referring briefly to FIG. 4, an example of a patch 420 extracted from an image volume 400 is shown, with a matrix size (voxel) of patch 420 in X and Y directions indicated on the X and Y axes. Image volume 400 is shown in three perspective views: a first perspective view 402 along the Y dimension (coronal view); a second perspective view 404 along the X dimension (sagittal view); and a third perspective view 406 along the Z dimension (axial view). In each of the three perspective views, a same anatomical structure 408 is shown. In first perspective view 402, a first bounding box 410 of patch 420 is shown in an x/z plane, where first bounding box 410 includes anatomical structure 408. In second perspective view 404, a second bounding box 412 of patch 420 is shown in a y/z plane, where second bounding box 412 also includes anatomical structure 408. In third perspective view 406, a third bounding box 414 of patch 420 is shown in a x/y plane, where third bounding box 414 also includes anatomical structure 408. Thus, bounding boxes 410, 412, and 414 define patch 420.

Returning to method 500, at 508, method 500 includes using a second trained model to customize the predicted beta value outputted by the neural network model, based on preferences of the operator. The second trained model may be a non-limiting example of beta customization model 310 of FIGS. 3A and 3C. The second trained model may also be a neural network model, or the second trained model may be a different type of model. For example, the second trained model may be a machine learning model, or a rules-based model implemented via a lookup table, or a curve-fitting model, or a different type of model. The second trained model may be trained on preference data collected from a plurality of operators of various PET imaging systems, as described in greater detail below in reference to FIG. 7. The second trained model may take as input one or more settings of the PET imaging system, the predicted beta value, and an identifier of the operator, and may output a customized beta value for the operator. The customized beta value may be different from the predicted beta value. For example, the customized beta value may take into consideration a preferred CNR of the operator with respect to a final reconstructed image, or a desired amount of noise.

At 510, method 500 includes reconstructing a second image volume from the raw image data using a second reconstruction method, where the second reconstruction method uses the customized beta value. The second reconstruction method may be different from the first reconstruction method. For example, the second reconstruction method may be a block sequential regularized expectation maximization (BSREM) method. The second reconstruction method may performed over a longer duration to increase a number of counts obtained, thereby increasing a quality of the second image volume. For example, the second image volume may have less noise, or a greater CNR than the first image.

In some embodiments, the first trained model may be used to predict distinct beta values for different portions of the first image volume located at different bed positions of the PET imaging system, as described above in reference to FIG. 3B. For example, the parameter recommendation system may divide the first image volume into a number of sections corresponding to the different bed positions. patches from each section of the number of sections may be inputted into the first trained model independently, to generate a predicted beta value for the corresponding bed position. The predicted beta values for each of the different bed positions may then each be inputted into the second trained model independently, to be customized by the second trained model. In such cases, an output of the parameter recommendation system may be a plurality of recommended beta values for a corresponding plurality of bed positions. The plurality of recommended beta values may subsequently be used in reconstructing the second image volume, where the second image volume may include sections corresponding to the bed positions that have been reconstructed with different recommended beta values. For example, a physician may wish to see the first portion of the second image volume with a first amount of noise; a second portion of the second image volume with a second amount of noise; a third portion of the second image volume with a third amount of noise; and so on.

At 512, method 500 includes displaying the second image volume on the display device to be viewed by the operator, and/or storing the second image volume in the PET imaging system (e.g., in image database 214 of FIG. 2) for viewing at a later time.

At 514, method 500 includes receiving feedback from the operator with respect to the second image volume. The feedback may be stored in a database of user preferences (e.g., database 380 of FIG. 3C) and used to further train or refine the second trained model (e.g., the beta customization model) to more accurately customize the second beta value to the operator. The feedback may be provided on the second image volume as a whole, or on 2D images extracted from the second image volume. For example, the operator may submit a first feedback on a first 2D image of a first portion of the second image volume, the first portion corresponding to a first bed position and having a first beta value; and the operator may submit a second feedback on a second 2D image of a second portion of the second image volume, the second portion corresponding to a second bed position and having a second beta value.

As an example of how method 500 may be used in practice, a physician may wish to perform a PET exam on a subject. The physician may select a desired scanning protocol for the exam, based on a desired clinical task. The physician may acquire raw image data from the subject based on the scanning protocol, and reconstruct the first image (volume) based on a set of default initial parameters. For example, a regularization term may not be specified. Based on the set of default initial parameters, the first image may have a first amount of noise and a first CNR. The first amount of noise may be higher than desired and the first CNR may be lower than desired as a result of the fast reconstruction. The operator may intend to perform a second, slower reconstruction of the raw image data to generate a second image with a second amount of noise and a second CNR, where the second amount of noise is less than the first amount of noise and the second CNR is greater than the first CNR. To generate the second image, the second, slower reconstruction of the raw image data may be performed using a recommended beta value. To determine the recommended beta value, the operator may input the first image into the parameter recommendation system. The parameter recommendation system may input the first image into the first trained model (e.g., the beta prediction model). The first trained model may predict an appropriate beta value for the second reconstruction method. For example, the predicted appropriate beta value may be 850.

The predicted appropriate beta value may be inputted into the second trained model, along with an identifier of the physician retrieved from the PET imaging system and one or more settings of the PET system selected by the physician. The second trained model may be implemented as a reference table, where the reference table may map the predicted appropriate beta value to a customized beta value for the physician. For example, the customized beta value may be 875, which may reflect a preference on the part of the physician for less noise in the second image.

The customized beta value may be used to configure the PET imaging system for performing the second reconstruction. In some embodiments, the PET imaging system may be configured to use the customized beta value and the second reconstruction may be performed without manual input from the physician. In other embodiments, the parameter recommendation system may be separate from the PET imaging system, and the parameter recommendation system may display the recommended (e.g., customized) beta value to the physician. The physician may then manually configure the PET imaging system to perform the second reconstruction. Once the second reconstruction is performed, a final reconstructed image may be generated (e.g., final reconstructed image 314), and displayed on the display device.

In this way, an appropriate beta value may be determined and used to reconstruct an image with a desired noise characteristics, where the desired noise characteristics is personalized for the physician. Rather than selecting the appropriate beta value based on operator experimentation, the parameter recommendation system predicts and customizes the appropriate beta value based on stored user preference data of the physician, resulting in a configuration of the PET imaging system that produces a final reconstructed image tailored to the physician. The appropriate beta value may be determined quickly, based on a precursor image reconstructed with a test or preset beta value. To perform continuous training, the end user (physician) may also be asked to provide responses to a survey that can inform whether the selected parameters were sufficient for the exam, or, for instance, if more contrast enhancement or more noise reduction would be preferred.

Referring now to FIG. 6, an exemplary method 600 is shown for training a beta prediction model of a parameter recommendation system for a PET imaging system, such as such as beta prediction model 309 of parameter recommendation system 308 of FIGS. 3A and 3C. In various embodiments, the beta prediction model may be a neural network model based on a CNN, such as a very deep convolutional (VGG) network. Method 600 may be carried out by a training module and/or a neural network module of the parameter recommendation system, such as training module 210 and/or neural network module 208 of parameter recommendation system 202. One or more instructions of method 600 may be executed by a processor of the parameter recommendation system (e.g., processor 204). Some instructions of method 500 may be performed manually by the operator.

Method 600 begins at 602, where method 600 includes collecting raw image data from a plurality of PET exams performed at various sites. Raw image data of various subjects of different sizes, ages, and other characteristics may be collected to ensure a balanced image population, including image data of healthy subjects and images of subjects with lesions or tumors. The data may further include scans taken with different tracers, and with patients in different disease states.

At 604, method 600 includes reconstructing images from the raw image data with various noise levels, using different beta values to control an amount of noise present in each image. Raw image data from each PET exam may be reconstructed a plurality of times into a respective plurality of 3D PET images.

For example, raw image data from a first PET exam may be reconstructed a first time, with a first beta value; a second time, with a second, smaller beta value; a third time, with a third beta value, the third beta value smaller than the first and second beta values; and a fourth time, with a fourth beta value, the fourth beta value smaller than the first, second, and third beta values. As a result, four 3D PET images of a same subject/anatomy may be generated, where each of the 3D PET images has a different level of noise. Specifically, a first 3D PET image may have a first amount of noise; a second 3D PET image may have a greater amount of noise than the first 3D PET image; a third 3D PET image may have a greater amount of noise than the first and second 3D PET images; and a fourth 3D PET image may have a greater amount of noise than the first, second, and third 3D PET images. The beta values may be manually selected by human experts to generate images of a high quality, where a CNR is maximized for each noise level.

At 606, method 600 includes labeling the 3D PET images reconstructed at 604 with a corresponding beta value used for reconstruction. In other words, in the example above, the first 3D PET image may be labeled with the first beta value; the second 3D PET image may be labeled with the second beta value; and so on. In some embodiments, the 3D PET images may be labeled with other, additional data, such as, for example, body mass index (BMI), injected dose, noise equivalent count rate (NECR), or other data relevant to image quality.

At 608, method 600 includes extracting and labeling a plurality of 3D image patches from the reconstructed 3D PET images, as described above in reference to FIG. 4. From each image volume, various 3D patches including various anatomical structures or features may be extracted. Each extracted patch may be assigned the beta value associated with the corresponding 3D PET image. Each extracted patch and an associated beta value may comprise a training pair used to train the beta prediction model.

At 610, method 600 includes training the beta prediction model to predict a beta value for each patch, using the labeled beta value as ground truth data. The beta prediction model may be a CNN including one or more convolutional layers, which in turn comprise one or more convolutional filters. The convolutional filters may comprise a plurality of weights, wherein the values of the weights are learned during a training procedure. The convolutional filters may correspond to one or more visual features/patterns, thereby enabling the CNN to identify and extract features from the patches.

Training the CNN may include iteratively inputting an input image of each training image pair (e.g., the patch) into an input layer of the CNN. In one embodiment, each voxel intensity value of the input image is inputted into a node of the input layer of the CNN. The CNN propagates the input image data from the input layer, through one or more hidden layers, until reaching an output layer of the CNN. In one embodiment, a first set of hidden layers perform feature extraction (e.g., noise, edges, objects) from the image input data, and a second set of hidden layers perform a regression that maps the extracted features to a single scalar value, which may be outputted by the CNN as a predicted beta value.

The CNN may be configured to iteratively adjust one or more of the plurality of weights of the CNN in order to minimize a loss function, based on a difference between the predicted beta value and the target beta value of the training pair. The difference (or loss), as determined by the loss function, may be back-propagated through the CNN to update the weights (and biases) of the hidden (convolutional) layers. In some embodiments, back propagation of the loss may occur according to a gradient descent algorithm, wherein a gradient of the loss function (a first derivative, or approximation of the first derivative) is determined for each weight and bias of the deep neural network. Each weight (and bias) of the CNN is then updated by adding the negative of the product of the gradient determined (or approximated) for the weight (or bias) with a predetermined step size. Updating of the weights and biases may be repeated until the weights and biases of the CNN converge, or the rate of change of the weights and/or biases of the CNN for each iteration of weight adjustment are under a threshold.

In order to avoid overfitting, training of the CNN may be periodically interrupted to validate a performance of the CNN on the training pairs. Training of the CNN may end when a performance of the CNN on a set of test pairs converges (e.g., when an error rate on the test set converges on or within a threshold of a minimum value). In this way, the CNN may be trained to predict the beta values associated with the input images during an inference stage, as described above in reference to FIG. 5. After the CNN has been trained and validated, the trained CNN may be stored in a memory of the parameter recommendation system for use in future PET exams. For example, the trained CNN may be stored in an inference module of the parameter recommendation system (e.g., inference module 212).

FIGS. 8A and 8B show example output results of training the beta prediction model. Referring to FIG. 8A, a beta value comparison chart 800 includes two rows. In a first row 830, four examples of input into the beta prediction model (e.g., training pairs) are shown, including input images and corresponding target beta values for an exam of a given duration. Exams are performed at four different durations, corresponding to four columns, where a different number of counts are obtained at each duration. A first column 840 shows images from a first exam with a first, longest duration, where a target number of counts is obtained. A second column 842 shows images from a second exam with a second, shorter duration, where the images are generated using 75% of the counts of the first exam. A third column 844 shows images from a third exam with a third duration, the third duration less than the second duration, where the images are generated using 50% of the counts of the first exam. A fourth column 846 shows images from a fourth exam with a fourth duration, the fourth duration less than the third duration, where the images are generated using 25% of the counts of the first exam. In this way, images for the training pairs are generated from images with varying amounts of noise. It should be appreciated that the relative count densities described herein are for illustrative purposes, and the above may not represent an optimal strategy for choosing different count densities.

During reconstruction, different beta values are used for the different input images, to reduce the amount of noise visible in the images. First input image 802 is reconstructed with a first beta value of 550; second input image 804 is reconstructed with a second beta value of 550; third input image 806 is reconstructed with a third beta value of 750; and fourth input image 808 is reconstructed with a fourth beta value of 900. Thus, as the number of counts used to generate an image decreases, the beta value may be increased to compensate. A result of increasing the beta value to compensate is that a difference in resolution and CNR between first input image 802, second input image 804, third input image 806, and fourth input image 808 may be reduced.

In various embodiments, one or more patches may be extracted from the input images and inputted into the beta prediction model, along with the target beta for the corresponding input image. In FIG. 8A, a first patch 835 and a second patch 836 are shown, which are extracted from each of input images 802, 804, 806, and 808. In other embodiments, different patches, and/or a different number of patches may be extracted and inputted into the beta prediction model.

A second row 832 shows an exemplary series of images reconstructed using beta values outputted by the beta prediction model. In other words, a first set of raw image data may be initially reconstructed to generate first input image 802, using the first beta value of 550. A first training pair including patch 835 and the target beta value of 550 may be inputted into the beta prediction model. Based on patch 835 and the first (target) beta value of 550, the beta prediction model may predict a beta value of 563. A first output image 812 may then be reconstructed from the first set of raw image data using the predicted beta value of 563.

Similarly, a second set of raw image data may be initially reconstructed to generate second input image 804, using the second beta value of 550. A training pair including patch 837 and the target beta value of 550 may be inputted into the beta prediction model. Based on patch 837 and the second (target) beta value of 550, the beta prediction model may predict a beta value of 623. A second output image 814 may then be reconstructed from the second set of raw image data using the predicted beta value of 623.

FIG. 8B shows a bed position chart 850 with exemplary outputs of the beta prediction model, where beta values are predicted for a plurality of bed positions. As with FIG. 8A, exams are performed at four different durations, corresponding to four columns, where a different number of counts are obtained at each duration. A first column 852 shows an image 870 reconstructed from a first exam with a first, longest duration, where a target number of counts is obtained. A second column 854 shows an image 872 from a second exam with a second, shorter duration, where image 870 is generated using 75% of the counts of the first exam. A third column 856 shows an image 874 from a third exam with a third duration, the third duration less than the second duration, where image 874 is generated using 50% of the counts of the first exam. A fourth column 858 shows an image 876 from a fourth exam with a fourth duration, the fourth duration less than the third duration, where image 876 is generated using 25% of the counts of the first exam.

Six bed positions are depicted in FIG. 8B, including a first bed position 860, a second bed position 862, a third bed position 864, a fourth bed position 866, a fifth bed position 868, and a sixth bed position 869. For each of the bed positions, a corresponding beta value may be predicted by the beta prediction model. In other words, image data (e.g., an extracted patch) corresponding to first bed position 860 of image 870 may be inputted into the beta prediction model, and based on the image data, the beta prediction model may output a bed 1 predicted beta value (e.g., bed 1 beta value 362 of FIG. 3B) of 685; image data corresponding to second bed position 862 of image 870 may be inputted into the beta prediction model, and based on the image data, the beta prediction model may output a bed 2 predicted beta value (e.g., bed 2 beta value 363 of FIG. 3B) of 530; image data corresponding to third bed position 864 of image 870 may be inputted into the beta prediction model, and based on the image data, the beta prediction model may output a bed 3 predicted beta value (e.g., bed 3 beta value 364 of FIG. 3B) of 525; and so on for bed positions 866, 868, and 869, and for images 872, 874, and 876.

In some embodiments, the predicted beta values for each bed position may be averaged to generate an overall beta value for an image (e.g., recommended beta value 311 of FIG. 3B). For example, the predicted beta values for each bed position of image 870 may be averaged to obtain an overall beta value of 564 for image 870; the predicted beta values for each bed position of image 872 may be averaged to obtain an overall beta value of 616 for image 872; the predicted beta values for each bed position of image 874 may be averaged to obtain an overall beta value of 731 for image 874; and the predicted beta values for each bed position of image 876 may be averaged to obtain an overall beta value of 888 for image 876. The overall beta values may be used to perform a second reconstruction of the corresponding image data, or during the second reconstruction, the individual beta values predicted for each bed position may be used to generate an image with different noise profiles at different bed positions.

It should be appreciated that for very large axial FOV systems (e.g., total body PET) there may not be “bed positions” as such, but a correct beta value may need to be modulated with respect to axial location to account for greater differences in per-slice sensitivity.

Referring now to FIG. 7, an exemplary method 700 is shown for training a parameter customization model of a parameter recommendation system for a PET imaging system, such as such as beta customization model 310 of parameter recommendation system 308 of FIGS. 3A and 3C. In one embodiment, the beta customization model is a machine learning model, or rules-based model implemented as a lookup table. In other embodiments, the beta customization model may be a different type of model. Method 700 may be carried out by a training module of the parameter recommendation system, such as training module 210 of parameter recommendation system 202. One or more instructions of method 700 may be executed by a processor of the parameter recommendation system (e.g., processor 204). Some instructions of method 500 may be performed manually by human experts (e.g., physicians or operators of PET imaging systems).

Method 700 begins at 702, where method 700 includes receiving labeled image volumes with different noise levels reconstructed using different beta values. The labeled image volumes may be generated as described above in relation to FIGS. 8A and 8B, by performing reconstructions with different beta values on raw image data collected from different exams. For example, the labeled image volumes with different counts may be generated by performing exams of different durations, or different doses, or in another way. In various embodiments, the labeled image volumes may be the same labeled image volumes used to train the beta prediction model, as described above in reference to FIG. 6 and referenced during image dataset acquisition block 374 of FIG. 3C.

At 704, method 700 includes extracting sets of 2D images from the labeled image volumes with varying levels of noise and beta values, for conducting user preference analysis sessions. The extracted sets may be associated with a particular exam and image volume. The extracted sets may also be associated with a particular scanned subject.

For example, four exams may be performed on a same scanned subject at four different durations, as described above in reference to FIG. 8A. in some embodiments, the four exams may be performed on the same scanned subject in one sitting, to ensure a maximal correlation between image data acquired during the four exams. A first set of 2D images may be extracted from a first image volume generated from a first exam; a second set of 2D images may be extracted from a second image volume generated from a second exam, a third set of 2D images may be extracted from a third image volume generated from the third exam, and a fourth set of 2D images may be extracted from a fourth image volume generated from the fourth exam.

Images may then be paired or grouped across the first, second, third, and fourth sets of 2D images, to generate pairings or groupings of 2D images of different noise/beta levels. For example, a first image selected from the first set of 2D images may be paired with a second image selected from the second set of 2D images to generate a first image pairing, where the first image of the second image include a same anatomical region from a same scanned subject. The first image selected from the first set of 2D images may then be paired with a third image selected from the third set of 2D images to generate a second image pairing; the first image may be paired with a fourth image selected from the fourth set of 2D images to generate a third image pairing; the second image may be paired with the third image to generate a fourth image pairing; and so on. In some embodiments, groupings of three, four, or more images may also be grouped in this manner. In this way, a plurality of pairings or groupings of 2D images may be generated, where the images included in the pairings or groupings have different noise levels and are reconstructed with different beta values.

At 706, method 700 includes displaying the plurality of pairings or groupings of 2D images to a plurality of users of the parameter recommendation system and/or the PET imaging system. For example, the users may be physicians who wish to store their user preferences with respect to parameter settings (e.g., such as the beta value) of the PET imaging system. The plurality of pairings or groupings may be displayed to the users sequentially one by one. The users may be prompted to rate the images of each pairing or grouping, and/or submit a preference for one image of each pairing or grouping over one or more other images of the pairing or grouping. Each image of the pairing or grouping may have the same or similar count densities, such that a preference for optimal settings for a given set of data may be determined (e.g., if the pairs include different count densities, the preference will almost always be for a longer-duration scan).

At 708, method 700 includes receiving the preferences and/or ratings with respect to the images of different noise/beta values from the users, and storing the preferences and/or ratings in a preferences database of the parameter recommendation system (e.g., preferences database 216 of FIG. 2).

At 710, method 700 includes performing content filtering (e.g., for a given user) and/or collaborative filtering (e.g., for a plurality of users) on the stored preference data. For example, one or more clustering algorithms may be used to classify users with similar preferences into groups.

At 712, method 700 includes training the beta customization model to output beta values preferred by individual users and/or groups of users for images with varying amounts of noise. In some embodiments, the beta customization model may take as input a first beta value predicted for a user by a beta prediction model (e.g., beta prediction model 309 of FIGS. 3A and 3C), and the beta customization model may output a customized beta value for the user based on the preference data of the user accessed from the preferences database. In other words, the beta customization model may map a beta value predicted for a certain scanning protocol, scanner, and patient to a corresponding personalized beta value reflecting the individual preferences of the user. The beta customization model may be implemented in various ways, including non-linear curve fitting, machine learning, lookup table, etc. Inputs into the beta customization model may also include other information related to the scan, such as an injection dose, a patient weight, or other data.

Thus, a robust, hybrid parameter recommendation system is proposed herein that relies on a plurality of models to recommend suggested settings for parameters of an imaging system, such as a PET imaging system. The parameter recommendation system may include different models that are trained to recommend settings for different parameters. The models may include a parameter prediction model, which may be trained to predict an optimal parameter setting for acquiring or reconstructing an image volume, based on a noise level of a preliminary image acquired and reconstructed using default parameter settings. For example, the parameter may be a noise regularization value (e.g., a beta value) used during image reconstruction, or an acquisition time per bed during data acquisition. The parameter prediction model may be a CNN trained on labeled patches of image volumes.

The models may also include a parameter customization model, which may map a predicted parameter setting to a customized parameter setting preferred by an operator performing a scan. The parameter customization model may rely on user preference data collected experimentally from a plurality of users in a prior user preference analysis stage and stored in a database of the parameter recommendation system.

By combining a prediction model and a customization model in the hybrid parameter recommendation system, a user may be offered suggestions for parameter settings that are objectively appropriate based on factors including a type of scanner used, a scanning protocol, a clinical task, patient size, and/or or a presence of motion during acquisition, and subjectively appropriate based on an amount of noise that the user is willing to tolerate in a final reconstructed image. A novel approach for collecting the user preference data is disclosed, where users are presented pairs or groups of images of a same subject and anatomy with different noise levels due to being reconstructed with different parameters (e.g., beta values), and asked to rate one or more images as preferential. The preference data may further be processed, for example, to filter or classify users to different groups sharing similar preferences, which may be leveraged to improve parameter customization.

By using the parameter recommendation system, physicians performing scans may be provided an easy and efficient way of determining optimal settings of an imaging system that may lead to a highest possible image quality in a reconstructed image. In an alternative imaging system not including the parameter recommendation system, physicians may spend more time determining appropriate parameter settings by trial and error, and resulting images may be of a less-than-desired quality. As a result of using the parameter recommendation system, the physicians may fine tune images to include noise at a level or within a range desired by the physician, which may be different from a second level or second range desired by a different physician. Viewing images at a desired noise level may facilitate diagnosis, leading to improved patient outcomes. Further, the parameter recommendation system provided herein may result in more consistent image quality across patients of different demographics.

The technical effect of providing customized recommendations for parameter settings of an imaging system is that a quality of reconstructed images may be increased.

The disclosure also provides support for a method for a Positron Emission Tomography (PET) system, the method comprising: performing a first reconstruction of a first image volume using raw image data of the PET system without specifying a noise regularization parameter, extracting a plurality of patches of the first image volume, using a first trained model to predict a noise regularization parameter setting, based on the extracted patches, using a second trained model to customize the predicted noise regularization parameter setting for a user of the PET system, based on preference data of the user, performing a second reconstruction of a second image volume using the raw image data of the PET system and the customized, predicted noise regularization parameter setting, the second image volume having a higher image quality than the first image volume, and displaying the second image volume on a display device of the PET system. In a first example of the method, the method further comprises: reconstructing the first image volume using fast ordered subsets expectation maximization (OSEM) reconstruction. In a second example of the method, optionally including the first example, the first trained model is a convolutional neural network (CNN) trained on a plurality of patches extracted from image volumes having different noise levels, each patch of the plurality of patches labeled with a target noise regularization parameter setting. In a third example of the method, optionally including one or both of the first and second examples, the extracted patches are extracted from portions of the first image volume located at different bed positions of the PET system, and using the first trained model to predict the noise regularization parameter setting further comprises: using the first trained model to predict a bed noise regularization parameter for each bed position of the different bed positions, and one of: averaging the predicted bed noise regularization parameter settings to generate the second noise regularization parameter setting, and using the bed noise regularization parameters for each bed position in the second reconstruction. In a fourth example of the method, optionally including one or more or each of the first through third examples, the preference data of the user is based on ratings of the user of images of images having different noise levels and noise regularization parameters, that are displayed to the user in pairings or groupings. In a fifth example of the method, optionally including one or more or each of the first through fourth examples, for a first operator of a PET imaging system, the second reconstruction of the second image volume is performed with a first noise regularization value customized for the first operator, and the second image volume is displayed on the display device with a first CNR, and for a second operator of the PET imaging system, the second reconstruction of the second image volume is performed with a second noise regularization value customized for the second operator, and the second image volume is displayed on the display device with a second CNR, the second CNR different from the first CNR.

The disclosure also provides support for a hybrid recommendation system (RS) for recommending a parameter setting for acquiring and/or reconstructing an image via an imaging system, the hybrid RS comprising: a first model trained to predict a parameter setting based on a preliminary reconstructed image, and a second model trained to customize the predicted parameter setting based on a preference of a user of the hybrid RS. In a first example of the system, the imaging system includes a Positron Emission Tomography (PET) system, and the parameter setting is a setting for a noise regularization parameter used for reconstructing the image. In a second example of the system, optionally including the first example, the imaging system includes a Positron Emission Tomography (PET) system, and the parameter setting is an acquisition time per bed used for acquiring the image. In a third example of the system, optionally including one or both of the first and second examples, the first model is a convolutional neural network (CNN) trained on a plurality of patches extracted from image volumes including different noise levels, each patch of the plurality of patches labeled with a target noise regularization parameter setting. In a fourth example of the system, optionally including one or more or each of the first through third examples, the image volumes including different noise levels are generated by performing a plurality of scans of an anatomy of a subject, each scan having a different noise regularization parameter setting. In a fifth example of the system, optionally including one or more or each of the first through fourth examples, the second model is one of a lookup table a machine learning model, and a curve-fitting model. In a sixth example of the system, optionally including one or more or each of the first through fifth examples, the second model maps the predicted parameter setting to a customized parameter setting for the user, based on preference data of the user collected from user ratings of images reconstructed with a range of noise levels. In a seventh example of the system, optionally including one or more or each of the first through sixth examples, the preference data is filtered using content and collaborative filtering. In a eighth example of the system, optionally including one or more or each of the first through seventh examples, the preference data is used to classify the user to a group of users with similar noise preferences.

The disclosure also provides support for a Positron Emission Tomography (PET) imaging system, comprising: a processor and a non-transitory memory including instructions that when executed, cause the processor to: in a first, training stage, train a customization model to learn preferences of a user of the PET imaging system based on ratings received from the user for a plurality of images, the plurality of images reconstructed using a range of noise regularization parameters, and in a second, inference stage: input a noise regularization parameter into the trained customization model, receive a customized noise regularization parameter as an output of the trained customization model, reconstruct an image volume from raw image data using the customized noise regularization parameter, and display the image volume on a display device of the PET imaging system. In a first example of the system, the noise regularization parameter inputted into the trained customization model is predicted by a prediction model of the PET imaging system. In a second example of the system, optionally including the first example, the customization model is one of a machine learning model, a rules-based model, a curve-fitting model, or a lookup table. In a third example of the system, optionally including one or both of the first and second examples, the plurality of images are reconstructed using a range of noise regularization parameters by performing a plurality of PET exams on a same anatomical region of a same scanned subject, and reconstructing images from each PET exam of the plurality of PET exams using a different noise regularization parameter. In a fourth example of the system, optionally including one or more or each of the first through third examples, the ratings are received from the user by displaying a plurality of pairings or groupings of images reconstructed with the different noise regularization parameters and different noise levels to the user, and receiving indications of a preference of the user for one image of each pairing or grouping over other images of the pairing or grouping.

When introducing elements of various embodiments of the present disclosure, the articles “a,” “an,” and “the” are intended to mean that there are one or more of the elements. The terms “first,” “second,” and the like, do not denote any order, quantity, or importance, but rather are used to distinguish one element from another. The terms “comprising,” “including,” and “having” are intended to be inclusive and mean that there may be additional elements other than the listed elements. As the terms “connected to,” “coupled to,” etc. are used herein, one object (e.g., a material, element, structure, member, etc.) can be connected to or coupled to another object regardless of whether the one object is directly connected or coupled to the other object or whether there are one or more intervening objects between the one object and the other object. In addition, it should be understood that references to “one embodiment” or “an embodiment” of the present disclosure are not intended to be interpreted as excluding the existence of additional embodiments that also incorporate the recited features.

In addition to any previously indicated modification, numerous other variations and alternative arrangements may be devised by those skilled in the art without departing from the spirit and scope of this description, and appended claims are intended to cover such modifications and arrangements. Thus, while the information has been described above with particularity and detail in connection with what is presently deemed to be the most practical and preferred aspects, it will be apparent to those of ordinary skill in the art that numerous modifications, including, but not limited to, form, function, manner of operation and use may be made without departing from the principles and concepts set forth herein. Also, as used herein, the examples and embodiments, in all respects, are meant to be illustrative only and should not be construed to be limiting in any manner.

1. A method for a Positron Emission Tomography (PET) system, the method comprising:
performing a first reconstruction of a first image volume using raw image data of the PET system without specifying a noise regularization parameter;
extracting a plurality of patches of the first image volume;
using a first trained model to predict a noise regularization parameter setting, based on the extracted patches;
using a second trained model to customize the predicted noise regularization parameter setting for a user of the PET system, based on preference data of the user;
performing a second reconstruction of a second image volume using the raw image data of the PET system and the customized, predicted noise regularization parameter setting, the second image volume having a higher image quality than the first image volume; and
displaying the second image volume on a display device of the PET system.

2. The method of claim 1, further comprising reconstructing the first image volume using fast ordered subsets expectation maximization (OSEM) reconstruction.

3. The method of claim 1, wherein the first trained model is a convolutional neural network (CNN) trained on a plurality of patches extracted from image volumes having different noise levels, each patch of the plurality of patches labeled with a target noise regularization parameter setting.

4. The method of claim 3, wherein the extracted patches are extracted from portions of the first image volume located at different bed positions of the PET system, and using the first trained model to predict the noise regularization parameter setting further comprises:
using the first trained model to predict a bed noise regularization parameter for each bed position of the different bed positions; and one of:
averaging the predicted bed noise regularization parameter settings to generate the second noise regularization parameter setting; and
using the bed noise regularization parameters for each bed position in the second reconstruction.

5. The method of claim 1, wherein the preference data of the user is based on ratings of the user of images of images having different noise levels and noise regularization parameters, that are displayed to the user in pairings or groupings.

6. The method of claim 1, wherein:
for a first operator of a PET imaging system, the second reconstruction of the second image volume is performed with a first noise regularization value customized for the first operator, and the second image volume is displayed on the display device with a first CNR; and
for a second operator of the PET imaging system, the second reconstruction of the second image volume is performed with a second noise regularization value customized for the second operator, and the second image volume is displayed on the display device with a second CNR, the second CNR different from the first CNR.

7. A hybrid recommendation system (RS) for recommending a parameter setting for acquiring and/or reconstructing an image via an imaging system, the hybrid RS comprising:
a first model trained to predict a parameter setting based on a preliminary reconstructed image; and
a second model trained to customize the predicted parameter setting based on a preference of a user of the hybrid RS.

8. The hybrid RS of claim 7, wherein the imaging system includes a Positron Emission Tomography (PET) system, and the parameter setting is a setting for a noise regularization parameter used for reconstructing the image.

9. The hybrid RS of claim 7, wherein the imaging system includes a Positron Emission Tomography (PET) system, and the parameter setting is an acquisition time per bed used for acquiring the image.

10. The hybrid RS of claim 8, wherein the first model is a convolutional neural network (CNN) trained on a plurality of patches extracted from image volumes including different noise levels, each patch of the plurality of patches labeled with a target noise regularization parameter setting.

11. The hybrid RS of claim 10, wherein the image volumes including different noise levels are generated by performing a plurality of scans of an anatomy of a subject, each scan having a different noise regularization parameter setting.

12. The hybrid RS of claim 7, wherein the second model is one of a lookup table a machine learning model, and a curve-fitting model.

13. The hybrid RS of claim 7, wherein the second model maps the predicted parameter setting to a customized parameter setting for the user, based on preference data of the user collected from user ratings of images reconstructed with a range of noise levels.

14. The hybrid RS of claim 13, wherein the preference data is filtered using content and collaborative filtering.

15. The hybrid RS of claim 13, wherein the preference data is used to classify the user to a group of users with similar noise preferences.

16. A Positron Emission Tomography (PET) imaging system, comprising:
a processor and a non-transitory memory including instructions that when executed, cause the processor to:
in a first, training stage, train a customization model to learn preferences of a user of the PET imaging system based on ratings received from the user for a plurality of images, the plurality of images reconstructed using a range of noise regularization parameters; and
in a second, inference stage:
input a noise regularization parameter into the trained customization model;
receive a customized noise regularization parameter as an output of the trained customization model;
reconstruct an image volume from raw image data using the customized noise regularization parameter; and
display the image volume on a display device of the PET imaging system.

17. The PET imaging system of claim 16, wherein the noise regularization parameter inputted into the trained customization model is predicted by a prediction model of the PET imaging system.

18. The PET imaging system of claim 16, wherein the customization model is one of a machine learning model, a rules-based model, a curve-fitting model, or a lookup table.

19. The PET imaging system of claim 16, wherein the plurality of images are reconstructed using a range of noise regularization parameters by performing a plurality of PET exams on a same anatomical region of a same scanned subject, and reconstructing images from each PET exam of the plurality of PET exams using a different noise regularization parameter.

20. The PET imaging system of claim 16, wherein the ratings are received from the user by displaying a plurality of pairings or groupings of images reconstructed with the different noise regularization parameters and different noise levels to the user, and receiving indications of a preference of the user for one image of each pairing or grouping over other images of the pairing or grouping.
